(19) United States
(12) Patent Application Publication
(10) Pub. No.: US 2006/0257421 A1
(43) Pub. Date: Nov. 16, 2006

(54) Pneumolysin derivatives

(76) Inventors: Martina Ochs, Toronto (CA); Fotula Fegaras, Richmond Hill (CA); Shaojiu Wang, Toronto (CA); Bruce Warner Carpick, Mississauga (CA); Wei Wei Qi, Bethlehem, PA (US); Tao Yuan, Oakville (CA); Lisa E. Myers, Rockwood (CA); James Tartaglia, Aurora (CA); Davinder Chawla, Thornhill (CA); Roy Persson, Toronto (CA); Olive James, Toronto (CA); Kimberly Matheson, Barrie (CA); Artur Pedyczak, Pickering (CA); Amanda Millar, Toronto (CA)

Correspondence Address: Reza Yacoob;Sanofi Pasteur Limited, 1755 Steeles Avenue West, Toronto, ON, M2R3T4, CA

(21) Appl. No.: 11/055,918

(22) Filed: Feb. 11, 2005

Related U.S. Application Data
(60) Provisional application No. 60/544,600, filed on Feb. 13, 2004.

(51) Int. Cl. C07K 14/315; C07H 21/04; C12P 21/06; A61K 39/02; C12N 1/21

(52) U.S. Cl. 424/190.1; 435/69.1; 435/320.1; 435/252.3; 530/350; 536/23.7

(57) ABSTRACT

The present invention relates to vaccines against bacterial pathogens. In particular, the invention relates to modified epitopes of bacterial antigens.

### RELATED APPLICATIONS

This application claims priority to U.S. Ser. No. 60/______ filed Feb. 13, 2004.

### FIELD OF THE INVENTION

The present invention relates to vaccines against bacterial pathogens.

### BACKGROUND OF THE INVENTION

Pneumolysin (PLY) is a multifunctional pneumococcal virulence factor that appears to augment intrapulmonary growth and dissemination during early pathogenesis of Streptococcus pneumoniae infection. PLY is a multifunctional cytolysin produced by all clinical isolates (Rubins and Janoff, J Lab Clin Med (1998) 131:21-27). PLY lacks an N-terminal secretion signal, however release into the extracellular environment has been observed in late logarithmic growth phase and for some strains in earlier growth stages (Balachandran et al. J Bac (2001) 183:3108-3116). Immunization with PLY evokes a protective immune response as demonstrated in numerous animal studies. However, the PLY derivatives described in the literature raised concerns about residual toxicity.

PLY is known to consist of four major structural domains (domains 1-4). Domain 1 (residues 6-21, 58-147, 198-243, 319-342) interacts with domain 1 of neighbouring PLY monomers with each other during oligomerization, which is required for PLY function (Rossjohn et al. Cell. 1997 May 30;89(5):685-92). Domain 2 functions as a hinge connecting domain 4 with domains 1 and 3. Domain 3 (residues 148-197, 244-318) inserts into the membrane during the oligomerization process (Rossjohn supra; Gilbert et al. J Mol Biol. 1998 Dec. 11;284(4):1223-37). Domain 4 (residues 360-469) has been shown to be essential for the initial binding to membrane cholesterol (Baba et al., BBRC 281: 37-44, 2001).

Modifications to pneumolysin have been previously reported in the literature (EP 449856B1; Baba, supra). Several attempts at detoxification through point mutations have also been reported in the literature (EP ⁴49856B1; Hill et al. Inf Imm (1994) 62:757-758; Korchev et al. Biochem J (1998) 329:571-577; Berry et al. Inf Imm (1995) 63:1969-1974). One of the most successful efforts was the construction of a triple mutant, Asp385Asn Cys428Gly Trp433Phe (PdT), that had no complement activating activity and its hemolytic activity was reduced to 0.001% of the wild-type activity. However, a S. pneumoniae strain carrying these mutations was not completely avirulent in mice compared to a PLY negative strain (Alexander et al. Microbial Pathogenesis (1998) 24:167-174).

Currently available PLY-based vaccines are associated with significant problems, including undesired toxicity. Those of skill in the art desire a PLY-based vaccine that is non-hemolytic in vitro, has a minimum of side effects in animals and humans, and causes the production of protective antibodies following immunization. It is most desirable that such antibodies neutralize wild-type PLY in vitro and protect in active (and passive) immunization studies against pneumococcal challenge. The present invention provides such as vaccine, as shown below.

### SUMMARY OF THE INVENTION

The present invention relates to recombinant DNA molecules encoding pneumolysin or derivatives thereof, polypeptides produced therefrom, and pharmaceutically acceptable compositions thereof.

### DETAILED DESCRIPTION

The present invention provides reagents and methodologies useful for treating and/or preventing bacterial infections. All references cited within this application are incorporated by reference.

In one embodiment, the present invention provides pneumolysin derivatives that are useful as vaccines against infection by Streptococcus pneumoniae. Examplary derivatives are shown in SEQ ID NOS.: 1-22. As described below, many variations of pneumolysin or the pneumolysin derivatives may be utilized in practicing the present invention. As described above, pneumolysis consists of four domains, Domains 1-4.. The present invention contemplates modifying these domains, isolating certain portions of the pneumolysin polypeptides (i.e.,. the PLY4 or PLY5 eptiopes), by removing one or more domains from the pneumolysin polypeptide, modifying one or more of the domains, or combining epitope isolation and/or domain removal and/or domain modification. In a preferred embodiment, Domain 4 is isolated and optionally modified as to its amino acid sequence and/or by joining Domain 4 to an internal antigenic or immunogenic amino acid sequence of Domains 1-3. For instance, the following non-limiting description of possible modifications of pneumolysin are provided: 1) Domain 4 without 5 to 10 C-terminal amino acids but plus PLY4 epitope and plus Asp385Asn; 2) Domain 4 plus PLY4 epitope plus D385N; 3) gene fusion of epitopes and/or domain 4 (Asp385Asn without C-terminal amino acids) to another antigen gene such as pspA, psaA, another pneumococcal antigen(s), non-toxic pertussis antigen(s) or genetically detoxified diphtheria toxin; 4) Domain 4 without 5 to 10 C-terminal amino acids; 5) Domain 4 without 5 to 10 C-terminal amino acids linked at its N- or C-terminus to the PLY4 epitope; 6) Domain 4 plus PLY4 epitope; 7) PLY4 and PLY5 epitopes (peptide) plus possibly other epitopes; 8) Domain 4; 9) Domains 1 to 3; 10) Domains 1 to 3 plus PLY5 epitope; 11) a segment of D1 (i.e., the first 21 amino acids) linked to Domain 4; 12) a segment of D2 (i.e., the first 20 amino acids) linked to Domain 4; 12) a histidine tag linked to Domain 4; 13) a pelB signal sequence linked to Domain 4; and, 14) internal deletions and/or modifications of any one or multiple domains. The different sequences may be linked to either the N-terminus or the C-terminus of the domain, for example, Domain 4. Other modifications of pneumolysin and/or equivalents that may be known to the skilled artisan due to the present description are also contemplated as part of the present invention.

In certain embodiments, the pneumolysin derivative is encoded by a nucleic acid sequence. Such nucleic acid sequence may be useful for expressing a pneumolysin derivative polypeptide or detecting the presence of a pneumolysin derivative in a sample, for example. The nucleic acid and polypeptide sequences described in this application may be substituted as desired. For instance, nucleotides contained within nucleic acid sequences may be substituted based on the degeneracy of the genetic code (i.e, consistent with the “Wobble” hypothesis). Where the nucleic acid is a recombinant DNA molecule useful for expressing a polypeptide in a cell, a Wobble-type substitution will result in the expression of a polypeptide with the same amino acid sequence as that originally encoded by the DNA molecule. In certain cases, however, substitutions may be conservative, or non-conservative, or any combination thereof. Conservative-amino acid modifications to the sequence of a polypeptide (and the corresponding modifications to the encoding nucleotides) may produce polypeptides having functional and chemical characteristics similar to those of a parental polypeptide. For example, a “conservative amino acid substitution” may involve a substitution of a native amino acid residue with a non-native residue such that there is little or no effect on the size, polarity, charge, hydrophobicity, or hydrophilicity of the amino acid residue at that position and, in particlar, does not result in decreased immunogenicity. Suitable conservative amino acid substitutions are shown in Table I.

TABLE I

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of the pneumolysin derivatives provided herein using well-known techniques. For identifying suitable areas of the molecule that may be changed without destroying biological activity (i.e., pore-forming activity, red blood cell (RBC) agglutination, RBC hemolysis, MHC binding, immunogenicity), one skilled in the art may target areas not believed to be important for that activity. For example, when pneumolysin derivatives with similar activities from the same species or from other species are known, one skilled in the art may compare the amino acid sequence of a polypeptide to such similar polypeptides. By performing such analyses, one can identify residues and portions of the molecules that are conserved. It will be appreciated that changes in areas of the molecule that are not conserved relative to such similar pneumolysin derivatives would be less likely to adversely affect the biological activity and/or structure of a polypeptide. Similarly, the residues required for binding to MHC are known, and may be modified to improve binding of pneumolysin antigenic sequences to MHC molecules. However, modifications resulting in decreased binding to MHC will not be appropriate in most situations. One skilled in the art would also know that, even in relatively conserved regions, one may substitute chemically similar amino acids for the naturally occurring residues while retaining activity. Therefore, even areas that may be important for biological activity or for structure may be subject to conservative amino acid substitutions without destroying the biological activity or without adversely affecting the structure of the pneumolysin derivative.

TABLE II
Types of Immunologic Adjuvants

| Type of | | |
|---|---|---|
| Adjuvant | General Examples | Specific Examples/References |
| Gel-type | Aluminum | (Aggerbeck and Heron, 1995) |
| | hydroxide/phosphate | |
| | (“alum adjuvants”) | |
| | Calcium phosphate | (Relyveld, 1986) |
| Microbial | Muramyl dipeptide | (Chedid et al., 1986) |
| | (MDP) | |
| | Bacterial | Cholera toxin (CT), E. coli labile |
| | exotoxins | toxin (LT)(Freytag and Clements, |
| | | 1999) |
| | Endotoxin-based | Monophosphoryl lipid A (MPL) |
| | adjuvants | (Ulrich and Myers, 1995) |
| | Other bacterial | CpG oligonucleotides (Corral and |
| | | Petray, 2000), BCG sequences |
| | | (Krieg, et al. Nature, 374: 576), |
| | | tetanus toxoid (Rice, et al. J. |
| | | Immunol., 2001, 167: 1558-1565) |
| Particulate | Biodegradable | (Gupta et al., 1998) |
| | Polymer | |
| | microspheres | |
| | Immunostimulatory | (Morein and Bengtsson, 1999) |
| | complexes (ISCOMs) | |
| | Liposomes | (Wassef et al., 1994) |
| Oil- | Freund's incomplete | (Jensen et al., 1998) |
| emulsion | adjuvant | |
| and | Microfluidized | MF59 (Ott et al., 1995) |
| surfactant- | emulsions | SAF (Allison and Byars, 1992) |
| based | | (Allison, 1999) |
| adjuvants | Saponins | QS-21 (Kensil, 1996) |
| Synthetic | Muramyl peptide | Murabutide (Lederer, 1986) |
| | derivatives | Threony-MDP (Allison, 1997) |
| | Nonionic block | L121 (Allison, 1999) |
| | copolymers | |
| | Polyphosphazene | (Payne et al., 1995) |
| | (PCPP) | |
| | Synthetic | Poly A:U, Poly I:C (Johnson, 1994) |
| | polynucleotides | |
| | Thalidomide | CC-4047/ACTIMID (J. Immunol., |
| | derivatives | 168(10): 4914-9) |

The pneumolyusin derivates described herein, having been fully described within this application, may also be used to generate antibodies for use in detection assays in the laboratory, for research, clinical monitoring, or immunotherapeutic purposes. Other uses would be apparent to one of skill in the art. The term “antibody” includes antibody fragments, as are known in the art, including Fab, Fab₂, single chain antibodies (Fv for example), humanized antibodies, chimeric antibodies, human antibodies, produced by several methods as are known in the art. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Harlow, et al. Using Antibodies: A Laboratory Manual, Portable Protocol No. 1, 1998; Kohler and Milstein, Nature, 256:495 (1975)); Jones et al. Nature, 321:522-525 (1986); Riechmann et al. Nature, 332:323-329 (1988); Presta (Curr. Op. Struct. Biol., 2:593-596 (1992); Verhoeyen et al. (Science, 239:1534-1536 (1988); Hoogenboom et al., J. Mol. Biol., 227:381 (1991); Marks et al., J. Mol. Biol., 222:581 (1991); Cole et al., Monoclonal Antibodies and Cancer Therapy, Alan R. Liss, p. 77 (1985); Boemer et al., J. Immunol., 147(1):86-95 (1991); Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995); as well as U.S. Pat. Nos. 4,816,567; 5,545,807; 5,545,806; 5,569,825; 5,625,126; 5,633,425; and, 5,661,016). Antibodies and their derivatives may be incorporated into compositions of the invention for use in vitro or in vivo.

A composition of the present invention may include a pneumolysin derivative, antibody against such a derivative, or the like, and is preferably pharmaceutically acceptable. A “pharmaceutically acceptable” composition is one that may be administered to a host such as an animal and will result in the desired effect (i.e., immunogenicity) with a minimum of associated side effects. Preferred embodiments of administratable compositions include, for example, nucleic acids or polypeptides in liquid preparations such as suspensions, syrups, or elixirs. Preferred injectable preparations include, for example, nucleic acids or polypeptides suitable for parental, subcutaneous, intradermal, intramuscular or intravenous administration such as sterile suspensions or emulsions. For example, a recombinant DNA molecule or polypeptide of the present invention may be in admixture with a suitable carrier, diluent, or excipient such as sterile water, physiological saline,. glucose or the like. The composition may also be provided in lyophilized form for reconstituting, for instance, in isotonic aqueous, saline buffer.

Administration of a composition of the present invention to a host may be accomplished using any of a variety of techniques known to those of skill in the art. The composition(s) may be processed in accordance with conventional methods of pharmacy to produce medicinal agents for administration to patients, including humans and other mammals. The pharmaceutical composition is preferably made in the form of a dosage unit containing a given amount of DNA, viral vector particles, polypeptide or peptide, for example. A suitable daily dose for a human or other mammal may vary widely depending on the condition of the patient and other factors, but, once again, can be determined using routine methods.

The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. In certain embodiments, a pharmaceutical composition is a composition comprising a therapeutically effective amount of a nucleic acid or polypeptide. The terms “effective amount” and “therapeutically effective amount” each refer to the amount of a nucleic acid or polypeptide used to induce or enhance an effective immune response. It is preferred that compositions of the present invention provide for the induction or enhancement of an immune response in a host which protects the host from the development of an infection or allows the host to eliminate an existing infection from the body.

For oral administration, the pharmaceutical composition may be of any of several forms including, for example, a capsule, a tablet, a suspension, or liquid, among others. Liquids may be administered by injection as a composition with suitable carriers including saline, dextrose, or water. The term parenteral as used herein includes subcutaneous, intravenous, intramuscular, intrastemal, infusion, or intraperitoneal administration. Suppositories for rectal administration of the drug can be prepared by mixing the drug with a suitable non-irritating excipient such as cocoa butter and polyethylene glycols that are solid at ordinary temperatures but liquid at the rectal temperature.

Pharmaceutical compositions comprising a nucleic acid or polypeptide of the present invention may take any of several forms and may be administered by any of several routes. In preferred embodiments, the compositions are administered via a parenteral route (intradermal, intramuscular or subcutaneous) to induce an immune response in the host. Alternatively, the composition may be administered directly into a lymph node (intranodal) or tumor mass (i.e., intratumoral administration). For example, the dose could be administered subcutaneously at days 0, 7, and 14.

While the compositions of the invention can be administered as the sole active pharmaceutical agent, they can also be used in combination with one or more other compositions or agents (i.e., other pneumolysin derivatives, co-stimulatory molecules, adjuvants). When administered as a combination, the individual components can be formulated as separate compositions administered at the same time or different times, or the components can be combined as a single composition.

Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

For topical administration, a suitable topical dose of a composition may be administered one to four, and preferably two or three times daily. The dose may also be administered with intervening days during which no dose is applied. Suitable compositions may comprise from 0.001% to 10% w/w, for example, from 1% to 2% by weight of the formulation, although it may comprise as much as 10% w/w, but preferably not more than 5% w/w, and more preferably from 0.1% to 1% of the formulation. Formulations suitable for topical administration include liquid or semi-liquid preparations suitable for penetration through the skin (e.g., liniments, lotions, ointments, creams, or pastes) and drops suitable for administration to the eye, ear, or nose.

The pharmaceutical compositions may also be prepared in a solid form (including granules, powders or suppositories). The pharmaceutical compositions may be subjected to conventional pharmaceutical operations such as sterilization and/or may contain conventional adjuvants, preservatives, stabilizers, wetting agents, emulsifiers, buffers etc. Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings. Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting sweetening, flavoring, and perfuming agents.

A kit comprising a composition of the present invention is also provided. The kit can include a separate container containing a suitable carrier, diluent, excipient, and/or an adjuvant. Additionally, the kit can include instructions for mixing or combining ingredients and/or administration, among other components as would be known in the art.

A better understanding of the present invention and of its many advantages will be had from the following examples, given by way of illustration.

### EXAMPLES

### Example 1

### Generation of Pneumolysin Derivatives

A. Construction of Plasmids.

The pneumolysin gene (ply) or fragments thereof were amplified by PCR from Streptococcus pneumoniae strain R36A or plasmids containing the cloned pneumolysin gene. Strain R36A was provided by David Briles, University of Alabama. Ply specific primers were designed according to the ply gene sequence of strain R6 (Hoskins et al., 2001). The PLY4 epitope (de los Toyos et al., 1996) was described as being located at amino acid 142, but the exact sequence was not defined. It was here assumed the epitope encompasses approximately amino acids 132 to 152 of wildtype pneumolysin.

The full-length pneumolysin gene was amplified from strain R36A using primers Spn 0001 and Spn 0002, the PCR product was digested with restriction enzymes NcoI and XhoI and cloned into plasmid pTrcK (Aventis Pasteur). Plasmid pTrcK is a kanamycin resistant derivative of plasmid pTrcHis2 (Invitrogen). The generated plasmid was named pBM46 and expression of the pneumolysin gene is under control of the trc-promoter. The DNA sequence of the amplicon was identical to the published pneumolysin sequence, encoding the deduced amino acid sequence shown in SEQ ID NO.: 1. The full-length gene was also amplified using primers Spn 0005 and Spn 0002. This PCR product was digested with enzymes BglII and XhoI and cloned into plasmid pET30b. The constructed plasmid was named pBM47 and encodes full-length pneumolysin with a N-terminal His-tag under control of the T7-promoter (SEQ ID NO.: 2).

The truncated pneumolysin gene containing domain 4 (D4) only, was amplified from strain R36A using primers Spn 0006 and Spn 0002, the PCR product was digested with restriction enzymes BglII and XhoI and cloned into plasmid pET30b (Novagen). In this construct, expression of D4 is under control of the T7-promoter and D4 contains a N-terminal Histidin-tag. The plasmid was named pBM49 and the DNA sequence of the amplicon was identical to the published sequence (deduced amino acid sequence SEQ ID NO.: 3). Domain 4 was also amplified from strain R36A with primers Spn 0007 and Spn 0002. This PCR product was digested with enzymes NcoI and XhoI and cloned into pTrcK. Expression of D4 is under control of the trc-promoter. The plasmid was named pBM52 and the DNA sequence of the amplicon was identical to the published sequence (deduced amino acid sequence SEQ ID NO.: 4).

The truncated pneumolysin gene containing domains 1-3 (D1-3) was amplified from strain R36A using primers Spn 0005 and Spn 0004, the PCR product was digested with restriction enzymes BglII and XhoI and cloned into plasmid pET30b (Novagen). In this construct, expression of D1-3 is under control of the T7-promoter and D1-3 contains a N-terminal Histidin-tag. This plasmid was named pBM48. The DNA sequence of the amplicon was identical to the published sequence (deduced amino acid sequence SEQ ID NO.: 5).

To eliminate pneumolysin mediated complement activation (Mitchell et al., 1991), amino acid Asp 385 was mutated to Asn. This was done by using the QuikChange site-directed mutagenesis kit (Stratagene). Plasmid pBM46, encoding the wild-type pneumolsyin gene, was chosen as template. Primers Spn 0012 and Spn 0013 were complementary to each other and both containing the mutation encoding Asp385Asn. The generated plasmid was named pBM50 (full-length pneumolysin gene Asp385Asn in pTrcK; deduced amino acid sequence shown in SEQ ID NO.: 6). Plasmid pBM64 was generated by PCR amplification of domain 4 D385N using primers Spn0006and Spn0002 and plasmid pBM50 as template. The amplicon was digested with enzymes BglII and XhoI and cloned into vector pET30b. Plasmid pBM65, carrying domain 4 with mutation D385N, was generated after amplification of the DNA fragment with plasmid pBM50 serving as template. Primers Spn0007 and Spn0002 were used in the PCR reaction and the PCR product was digested with enzymes NcoI and XhoI and cloned into the vector pTrcK.

Plasmid pBM53 contains D4 with mutation Asp385Asn and has the PLY4 epitope fused at the 3′ end of D4. The plasmid was constructed by PCR amplification of D4 with primers Spn 0007 and Spn 0015. pBM50 served as the template. Spn 0015 encoded the PLY4 epitope in its 5′ extension. The amplicon was digested with enzymes NcoI and XhoI and cloned into plasmid pTrcK. The DNA sequence of this modified D4 was confirmed (deduced amino acid sequence shown in SEQ ID NO.: 7). A similar construct was also generated with a N-terminal His-tag in plasmid pET30b. For this purpose, D4 was amplified with primers Spn 0006 and Spn 0015 using pBM50 as template. The amplicon was digested with enzymes BglII and XhoI, cloned into pET30b and the new plasmid was named pBM54. The expected DNA sequence of the modified D4 was confirmed (deduced amino acid sequence shown in SEQ ID NO.: 8). The domain 4 fragment from plasmid pBM53 was also cloned into vector pET15b (Novagen). For this, plasmid pBM53 was digested with the enzymes NcoI and XhoI and the domain 4 fragment was cloned into NcoI and XhoI sites of pET15b and this new plasmid was named pBM77. Similarly, the domain 4 fragment from plasmid pBM65 was transferred into vector pET15b, creating pBM86.

Plasmid pBM55 contains D4 with mutation Asp385Asn and the 10 C-terminal amino acids deleted fused to a sequence encoding the PLY4 epitope fused at the 3′ end of D4. In contrast pBM53 includes DNA encoding D4 with the 10 C-terminal amino acids deleted. This was achieved using primer Spn 0016. D4 was amplified from pBM50 by using primers Spn 0007 and Spn 0016. The amplicon was digested with enzymes NcoI and XhoI and cloned into plasmid pTrcK. The correctness of the expected DNA sequence was confirmed and the generated plasmid named pBM55 (SEQ ID NO.: 9). A similar construct was also generated with a N-terminal His-tag in plasmid pET30b. D4 was amplified with primers Spn 0006 and Spn 0016 using plasmid pBM50 as template. The amplicon was digested with enzymes BglII and XhoI and cloned into pET30b leading to plasmid pBM56. The correctness of the inserted DNA sequence was confirmed (deduced amino acid sequence shown in SEQ ID NO.: 10). Other sequences were also constructed as shown in SEQ ID NOS.: 11-29.

To remove the agglutinating activity of purified Domain 4 protein the pointmutation W433F was introduced into the full-length pneumolysin gene. Mutagenesis was performed with the QuikChange site-directed mutagenesis kit (Stratagene). Plasmid BM46 encoding the full-length pneumolysin gene served as template. Primers Spn0048 and Spn0049 were complementary to each other and contained both the mutation encoding W433F. The resulting plasmid was named pBM78 (SEQ ID NO.: 13). Plasmid pBM78 served subsequently as template to create plasmid pBM87 that contains the full-length pneumolysin gene with point mutations D385N and W433F. The point mutation D385N was introduced into this plasmid as described for the generation of pBM50.

Derivatives of plasmid pBM87 were generated. Plasmid pBM90 carries domain 4 with a N-terminal His-tag and pointmutations D385N W433F and was generated after PCR amplification of domain 4 with primers Spn0006 and Spn0002, digest of the amplicon with BglII and XhoI and cloning of the fragment into vector pET30b. Similarly, domain 4 of pBM87 was PCR amplified with primers Spn0007 and Spn0002. This amplicon was digested with NcoI and XhoI and cloned into vector pET15b and this plasmid was named pBM91.

B. Generation of Anti-Pneumolysin Antibodies.

Expression of His-tagged D4 and His-tagged D1-3 proteins was tested in E. coli BL21 (DE3). Both proteins were expressed upon IPTG (1 mM) induction and subsequently purified under denaturing conditions with the Ni-NTA purification kit from QIAGEN. The purified proteins were dialyzed against PBS and used for the immunization of rabbits. Two rabbits were immunized i.m. with the His-tagged D4 protein encoded by plasmid pBM49 at 20 μg/dose with Freund's adjuvant. The animals received 2 additional immunizations with incomplete Freund's adjuvant 2 and 4 weeks after the initial injection. Serum was collected 2 and 4 weeks after the last injection. The same was also done with the His-tagged D1-3 protein encoded by plasmid pBM48.

Antibodies were also generated against purified His-tagged Domain 4 protein encoded by plasmids pBM54 and pBM64. Both proteins were purified utilizing Ni-NTA facilitated purification. Typically, two rabbits were immunized with each protein at 5 μg/dose with Freund's adjuvant. The animals received 2 additional immunizations with incomplete Freund's adjuvant 2 and 4 weeks after the initial injection. Serum was collected 2 and 4 weeks after the last injection.

Domain 4 polypeptides were also expressed from pBM46, pBM91 and pBM86, purified, and used to immunize rabbits. In certain assays, the peptides 327811 (ECTGLAWEWWRTVYE) and 327815 (WDRNGQDLTAHFTT) were used as antigens. The results are discussed below.

C. Characterization of Anti-Pneumolysin Antibodies.

For polypeptides expressed from pBM48, pBM49, pBM54 and pBM64, the generation of antibodies was tested by western blotting. Anti-D1-3 antibodies reacted with a lysate of E. coli pBM46 expressing the wildtype pneumolysin protein and anti-D4 antibodies reacted with wildtype pneumolysin and a lysate of E. coli pBM52 expressing D4 only.

For polypeptides expressed from pBM46, pBM86 and pBM91, rabbit sera was tested by both ELISA and the IGEN competition assay using standard procedures to detect the presence of antibodies specific for known neutralizing epitopes of D4. It was observed that all rabbits immunized with pBM91 and pBM86 polypeptides produces IgG antibodies that bind to full length PLY in ELISA and IGEN assays with good serum titres. Competition IGEN assays confirmed the specificity of this binding for the D4 portion of full length PLY of good titres. Binding of antibody from individual anti-D4 sera to peptide 327815, compared to irrelevant control peptide CLP3620, indicated that multiple rabbits produced detectable antibody targeting the same amino acid sequence as the neutralizing mAb PLY7. Thus, immunization with truncated pneumolysin derivatives led to the production of antibodies reacting with wildtype pneumolysin.

D. Toxicity of Pneumolysin Derivatives.

The pneumolysin derivatives His-tagged Domain 4 Asp385Asn and His-tagged Domain 4 Asp385Asn+PLY4 were tested in a hemolysis assay. Briefly, purified His-tagged Domain 4 Asp385Asn and His-tagged Domain 4 Asp385Asn+PLY4 were separately diluted serially 1:2 in PBS in a 100 μl volume and mixed with 100 μl of a 1% Sheep Red Blood Cell (SRBC) solution. Incubation was allowed to proceed for one hour at 37° C., and hemolysis determined visually. It was observed that the derivatives were not hemolytic, but did cause SRBC agglutination. Minimal agglutinating protein concentration were 100.9 ng/100 μl 1% SRBC for His-tagged Domain 4 Asp385Asn and 39.7 ng/100 μl 1% SRBC for His-tagged Domain 4 Asp385Asn plus C-term PLY4.

Mutation of Domain 4 Trp433 to Phe was observed to significantly decrease agglutination. In one experiment, it was determined that the minimal agglutinating protein concentration for His-tagged Domain 4 Asp385Asn was 100.9 ng/100 μl 1% SRBC; for His-tagged Domain 4 Asp385Asn plus C-term PLY4, it was 39.7 ng/100 μl 1 % SRBC; and, for His-tagged Domain 4 Trp433Phe it was 6,973 ng/100 μl 1% SRBC.

The toxicity of the polypeptide expressed from pBM46, pBM86 and pBM91 were also tested. Each sample was diluted two-fold from neat, in a final volume of 100 ul PBS (phosphate buffered saline), in a 96 well plate. A volume of 100 ul of 1% sheep red blood cells in PBS was added to each well containing 100 ul of diluted sample. Plates were incubated at 37° C. for 1 hour. Wells were examined for the presence or absence of a cell pellet. Hemolytic titre was defined by the furthest dilution of sample at which hemolysis, and absence of a pellet, was observed. A titre of <1 meant that no hemolytic activity was detected in an undiluted sample. As shown in Table III below, it was determined that the pBM86 and pBM91 polypeptides have essentially no detectable hemolytic activity.

TABLE III

| | | | Hemolytic |
|---|---|---|---|
| Sample | Description | Concentration | titre* |
| (1) TY756 | pBM46, full-length PLY | 310.4 ug/ml | 16,384 |
| (2) TY1517 | pBM46, full-length PLY | 174.3 ug/ml | 16,384 |
| (3) TY1438 | pBM86, D4 D385N | 133 ug/ml | <1 |
| (4) TY832 | pBM86, D4 D385N | 194 ug/ml | <1 |
| (5) TY1234 | pBM91, D4 D385N W433F | 717 ug/ml | <1 |
| (6) TY1916 | pBM91, D4 D385N W433F | 279 ug/ml | <1 |

E. Immunization of Mammals

It may be desirable to immunize an animal with a DNA molecule a polypeptide or polypeptide in an amount sufficient to induce an immune response against the polypeptide (i.e., antibodies are produced). To accomplish this, the polypeptides are administered either without adjuvant or with an appropriate adjuvant such as AlPO₄ or Al(OH)₃. Administration of Domain 4 polypeptides (i.e., SEQ ID NOS.: 1-29) alone or in combination with other pneumococcal antigens (proteins and or polysaccharides), other non-pneumococcal proteins such as for example pertussis toxin. Preferably, administration is via the parenteral or intramuscular route using standard techniques. Typically, several doses administered (i.e., three doses and a booster).

For instance, mice were immunized using an interferon emulsion as an adjuvant, as shown in Table IV. In several cases, immunization with the polypeptide expressed from pBM91 (indicated as “D4” in the table) in combination with the intereferon emulsion adjuvant resulted in detectable anti-pneumolysin antibodies.

In another set of experiments, a sepsis animal model was tested. The data indicated that anti-PspA (anti-Rx1MI) antibodies do not protect mice against lethal S. pneumoniae challenge with this strain unless combined with anti-pBM91 antisera. Anti-pBM91 antibodies were observed to provide protection against lethal infection. It was also observed that passive transfer of anti-pBM91 antibodies alone delayed the time to death.

While the present invention has been described in terms of the preferred embodiments, it is understood that variations and modifications will occur to those skilled in the art. Therefore, it is intended that the appended claims cover all such equivalent variations that come within the scope of the invention as claimed.

!SEQUENCE LISTING
SEQ ID NO.: 1
Deduced amino acid sequence of pneumolysin encoded
by plasmid pBM46.
MANKAVNDFILANNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYDHQGKEVLTPKAWDRN
GQDLTAHFTTSIPLKGNVRNLSVKIRECTGLAWEWWRTVYEKTDLPLVRK
RTISIWGTTLYPQVEDKVEND*
SEQ ID NO.: 2
Deduced amino acid sequence of pneumlysin with
N-terminal His-tag (boxed) encoded by plasmid
pBM47. [symbol]
MANKAVNDFILAMN
YDKKKLLTHQGESIENRFIKEGNQLPDEFVVIERKKRSLSTNTSDISVTA
TNDSRLYPGALLVVDETLLENNPTLLAVDRAPMTYSIDLPGLASSDSFLQ
VEDPSNSSVRGAVNDLLAKWHQDYGQVNNVPARMQYEKITAHSMEQLKVK
FGSDFEKTGNSLDIDFNSVHSGEKQIQIVNFKQIYYTVSVDAVKNPGDVF
QDTVTVEDLKQRGISAERPLVYISSVAYGRQVYLKLETTSKSDEVEAAFE
ALIKGVKVAPQTEWKQILDNTEVKAVILGGDPSSGARVVTGKVDMVEDLI
QEGSRFTADHPGLPISYTTSFLRDNVVATFQNSTDYVETKVTAYRNGDLL
LDHSGAYVAQYYITWDELSYDHQGKEVLTPKAWDRNGQDLTAHFTTSIPL
KGNVRNLSVKIRECTGLAWEWWRTVYEKTDLPLVRKRTISIWGTTLYPQV
EDKVEND*
SEQ ID NO.: 3
Deduced amino acid sequence of domain 4 with a
N-terminal His-tag (boxed) encoded by pBM49. [symbol]
GDLLLDHSGAYVAQ
YYITWDELSYDHQGKEVLTPKAWDRNGQDLTAHFTTSIPLKGNVRNLSVK
IRECTGLAWEWWRTVYEKTDLPLVRKRTISIWGTTLYPQVEDKVEND*
SEQ ID NO.: 4
Deduced amino acid sequence of domain 4 encoded
by pBM52.
MGDLLLDHSGAYVAQYYUITWDELSYDHQGKEVLTPKAWDRNGQDLTAHF
TTSIPLKGNVRNLSVKIRECTGIIAWEWWRTVYEKTDLPLVRKRTISIWG
TTLYPQVEDKVEND*
SEQ ID NO.: 5
Deduced amino acid sequence of domain 1-3 with a
N-terminal His-tag (boxed) encoded by pBM48. [symbol]
MANKAVNDFILAMN
YDKKKLLTHQGESIENRFIKEGNQLPDEFVVIERKKRSLSTNTSDISVTA
TNDSRLYPGALLVVDETLLENNPTLLAVDRAPMTYSIDLPGLASSDSFLQ
VEDPSNSSVRGAVNDLLAKWHQDYGQVNNVPARMQYEKITAHSMEQLKVK
FGSDFEKTGNSLDIDFNSVHSGEKQIQIVNFKQIYYTVSVDAVKNPGDVF
QDTVTVEDLKQRGISAERPLVYISSVAYGRQVYLKLETTSKSDEVEAAFE
ALIKGVKVAPQTEWKQILDNTEVKAVILGGDPSSGARVVTGKVDMVEDLI
QEGSRFTADHPGLPISYTTSFLRDNVVATFQNSTDYVETKVTAYRN*
SEQ ID NO.: 6
Deduced amino acid sequence of pneumolysin with
mutation Asp385Asn (in bold) encoded by pBM50.
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYNHQGKEVLTPKAWDRN
GQDLTAHFTTSIPLKGNVRNLSVKIRECTGLAWEWWRTVYEKTDLPLVRK
RTISIWGTTLYPQVEDKVEND*
SEQ ID NO.: 7
Deduced amino acid sequence of domain 4 Asp385Asn
plus C-terminal PLY4 epitope (italics) encoded by
pBM53.
MGDLLLDHSGAYVAQYYITWDELSYNHQGKEVLTPKAWDRNGQDLTAHFT
TSIPLKGNVRNLSVKIRECTGLAWEWWRTVYEKTDLPLVRKRTISIWGTT
LYPQVEDKVENDHQDYGQVNNVPARMQYEKI*
SEQ ID NO.: 8
Deduced amino acid sequence of domain 4 Asp385Asn
plus C-terminal PLY4 epitope (italics) with a
N-terminal His-tag (boxed) encoded by pBM54. [symbol]
GDLLLDHSGAYVAQ
YYITWDELSYNHQGKEVLTPKAWDRNGQDLTAHFTTSIPLKGNVRNLSVK
IRECTGLAWEWWRTVYEKTDLPLVRKRTISIWGTTLYPQVEDKVENDHQD
YGQVNNVPARMQYEKI*
SEQ ID NO.: 9
Deduced amino acid sequence of domain 4 Asp385Asn
(bold), last 10 C-terminal amino acids deleted,
plus C-terminal PLY4 epitope (italics) encoded by
pBM55
MGDLLLDHSGAYVAQYYITWDELSYNHQGKEVLTPKAWDRNGQDLTAHFT
TSIPLKGNVRNLSVKIRECTGLAWEWWRTVYEKTDLPLVRKRTISIWGTT
LYWHQDYGQVNNVPARMQYEKIT
SEQ ID NO.: 10
Deduced amino acid sequence of domain 4 Asp385Asn
(bold), last 10 C-terminal amino acids deleted,
plus C-terminal PLY4 epitope (italics) with a N-
terminal His-tag (boxed) encoded by pBM56 [symbol]
GDLLLDHSGAYVAQ
YYITWDELSYNHQGKEVLTPKAWDRNGQDLTAHFTTSIPLKGNVRNLSVK
IRECTGLAWEWWRTVYEKTDLPLVRKRTISIWGTTLYWHQDYGQVNNVPA
RMQYEKIT
SEQ ID NO.: 11
Deduced amino acid sequence of pneumolysin with
mutation D385L (in bold) encoded by pBM61
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYLHQGKEVLTPKAWDRN
GQDLTAHFTTSIPLKGNVRNLSVKIRECTGLAWEWWRTVYEKTDLPLVRK
RTISIWGTTLYPQVEDKVEND*
SEQ ID NO.: 12
Deduced amino acid sequence of pneumolysin with
mutation D385E (in bold) encoded by pBM62
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYEHQGKEVLTPKAWDRN
GQDLTAHFTTSIPLKGNVRNLSVKIRECTGLAWEWWRTVYEKTDLPLVRK
RTISIWGTTLYPQVEDKVEND*
SEQ ID NO.: 13
Deduced amino acid sequence of pneumolysin with
mutation W433F (in bold) encoded by pBM78
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYDHQGKEVLTPKAWDRN
GQDLTAHFTTSIPLKGNVRNLSVKIRECTGLAFEWWRTVYEKTDLPLVRK
RTISIWGTTLYPQVEDKVEND*
SEQ ID NO.: 14
Deduced amino acid sequence of pneumolysin with
mutation D385N and W433L (in bold) encoded by
pBM80
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYNHQGKEVLTPKAWDRN
GQDLTAHFTTSIPLKGNVRNLSVKIRECTGLALEWWRTVYEKTDLPLVRK
RTISIWGTTLYPQVEDKVEND*
SEQ ID NO.: 15
Deduced amino acid sequence of pneumolysin with
mutation D385N and W433F (in bold) encoded by
pBM87
MANKAVNDFILAMNYDKKKLLTHQGESIENRFIKEGNQLPDEFVVIERKK
RSLSTNTSDISVTATNDSRLYPGALLVVDE 80
TLLENNPTLLAVDRAPMTYSIDLPGLASSDSFLQVEDPSNSSVRGAVNDL
LAKWHQDYGQVNNVPARMQYEKITAHSMEQ 160
LKVKFGSDFEKTGNSLDIDFNSVHSGEKQIQIVNFKQIYYTVSVDAVKNP
GDVFQDTVTVEDLKQRGISAERPLVYISSV 240
AYGRQVYLKLETTSKSDEVEAAFEALIKGVKVAPQTEWKQILDNTEVKAV
ILGGDPSSGARVVTGKVDMVEDLIQEGSRF 320
TADHPGLPISYTTSFLRDNVVATFQNSTDYVETKVTAYRNGDLLLDHSGA
YVAQYYITWDELSYNHQGKEVLTPKAWDRN 400
GQDLTAHFTTSIPLKGNVRNLSVKIRECTGLAFEWWRTVYEKTDLPLVRK
RTISIWGTTLYPQVEDKVEND* 471
SEQ ID NO.: 16
Deduced amino acid sequence of pneumolysin with
mutations D385N, W433F, and W435R (in bold)
encoded by pBM89
MANKAVNDFILAMNYDKKKLLTHQGESIENRFIKEGNQLPDEFVVIERKK
RSLSTNTSDISVTATNDSRLYPGALLVVDE 80
TLLENNPTLLAVDPAPMTYSIDLPGLASSDSFLQVEDPSNSSVRGAVNDL
LAKWHQDYGQVNNVPARMQYEKITAHSMEQ 160
LKVKFGSDFEKTGNSLDIDFNSVHSGEKQIQIVNFKQIYYTVSVDAVKNP
GDVFQDTVTVEDLKQRGISAERPLVYISSV 240
AYGRQVYLKLETTSKSDEVEAAFEALIKGVKVAPQTEWKQILDNTEVKAV
ILGGDPSSGARVVTGKVDMVEDLIQEGSRF 320
TADHPGLPISYTTSFLRDNVVATFQNSTDYVETKVTAYRNGDLLLDHSGA
YVAQYYITWDELSYNHQGKEVLTPKAWDRN 400
GQDLTAHFTTSIPLKGNVRNLSVKIRECTGLAFERWRTVYEKTDLPLVRK
RTISIWGTTLYPQVEDKVEND* 471
SEQ ID NO.: 17
Deduced amino acid sequence of pneumolysin domain
4 D385N plus C-terminal PLY4 epitope (italics)
encoded by plasmid pBM77
MGDLLLDHSGAYVAQYYITWDELSYNHQGKEVLTPKAWDRNGQDLTAHFT
TSIPLKGNVRNLSVKIRECTGLAWEWWRTVYEKTDLPLVRKRTISIWGTT
LYPQVEDKVENDHQDYGQVNNVPARMQYEKI*
SEQ ID NO.: 18
Deduced amino acid sequence of pneumolysin Domain
4 with mutation D385E (in bold) + PLY4
GDLLLDHSGAYVAQYYITWDELSYNHQGKEVLTPKAWDRNGQDLTAHFTT
SIPLKGNVRNLSVKIRECTGLAWEWWRTVYEKTDLPLVRKRTISIWGTTL
YPQVEDKVENDHQDYGQVNNVPARMQYEKI*
SEQ ID NO.: 19
Deduced amino acid sequence of pneumolysin Domain
4 with mutation W433F (in bold) + PLY4
GDLLLDHSGAYVAQYYITWDELSYDHQGKEVLTPKAWDRNGQDLTAHFTT
SIPLKGNVRNLSVKIRECTGLAFEWWRTVYEKTDLPLVRKRTISIWGTTL
YPQVEDKVENDWHQDYGQVNNVPARMQYEKIT
SEQ ID NO.: 20
Deduced amino acid sequence of pneumolysin Domain
4 with mutation D385N and W433L (in bold) + PLY4
GDLLLDHSGAYVAQYYITWDELSYNHQGKEVLTPKAWDRNGQDLTAHFTT
SIPLKGNVRNLSVKIRECTGLALEWWRTVYEKTDLPLVRKRTISIWGTTL
YPQVEDKVENDWHQDYGQVNNVPARMQYEKIT
SEQ ID NO.: 21
Deduced amino acid sequence of pneumolysin Domain
4 with mutation D385N and W433F (in bold) + PLY4
GDLLLDHSGAYVAQYYITWDELSYNHQGKEVLTPKAWDRNGQDLTAHFTT
SIPLKGNVRNLSVKIRECTGLAFEWWRTVYEKTDLPLVRKRTISIWGTTL
YPQVEDKVENDWHQDYGQVNNVPARMQYEKIT
SEQ ID NO.: 22
Deduced amino acid sequence of pneumolysin with
mutations D385N, W433F, and W435R (in bold) + PLY4
GDLLLDHSGAYVAQYYITWDELSYNHQGKEVLTPKAWDRNGQDLTAHFTT
SIPLKGNVRNLSVKIRECTGLAFERWRTVYEKTDLPLVRKRTISIWGTTL
YPQVEDKVENDWHQDYGQVNNVPARMQYEKI
SEQ ID NO.: 23
Deduced amino acid sequence of pneumolysin domain
4 D385N (in bold) with N-terminal His-tag encoded
by plasmid pBM64 [symbol]
GDLLLDHSGAYVAQ
YYITWDELSYNHQGKEVLTPKAWDRNGQDLTAHFTTSIPLKGNVRNLSVK
IRECTGLAWEWWRTVYEKTDLPLVRKRTISIWGTTLYPQVEDKVEND*
SEQ ID NO.: 24
Deduced amino acid sequence of pneumolysin domain
4 D385N (in bold) encoded by plasmid pBM65
MGDLLLDHSGAYVAQYYITWDELSYNHQGKEVLTPKAWDRNGQDLTAHFT
TSIPLKGNVRNLSVKIRECTGLAWEWWRTVYEKTDLPLVRKRTISIWGTT
LYPQVEDKVEND
SEQ ID NO.: 25
Deduced amino acid sequence of pneumolysin domain
4 D385N (in bold) encoded by plasmid pBM86
MGDLLLDHSGAYVAQYYITWDELSYNHQGKEVLTPKAWDRNGQDLTAHFT
TSIPLKGNVRNLSVKIRECTGLAWEWWRTVYEKTDLPLVRKRTISIWGTT
LYPQVEDKVEND
SEQ ID NO.: 26
Deduced amino acid sequence of pneumolysin W433F
(in bold) encoded by plasmid pBM78
MANKAVNDFILANNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYDHQGKEVLTPKAWDRN
GQDLTAHFTTSIPLKGNVRNLSVKIRECTGLAFEWWRTVYEKTDLPLVRK
RTISIWGTTLYPQVEDKVEND*
SEQ ID NO.: 27
Deduced amino acid sequence of pneumolysin D385N
W433F (both in bold) encoded by plasmid pBM87
MANKAVNDFILAMNYDKKKLLTHQGESIENRFIKEGNQLPDEFVVIERKK
RSLSTNTSDISVTATNDSRLYPGALLVVDETLLENNPTLLAVDRAPMTYS
IDLPGLASSDSFLQVEDPSNSSVRGAVNDLLAKWHQDYGQVNNVPARMQY
EKITAHSMEQLKVKFGSDFEKTGNSLDIDFNSVHSGEKQIQIVNFKQIYY
TVSVDAVKNPGDVFQDTVTVEDLKQRGISAERPLVYISSVAYGRQVYLKL
ETTSKSDEVEAAFEALIKGVKVAPQTEWKQILDNTEVKAVILGGDPSSGA
RVVTGKVDMVEDLIQEGSRFTADHPGLPISYTTSFLRDNVVATFQNSTDY
VETKVTAYRNGDLLLDHSGAYVAQYYITWDELSYNHQGKEVLTPKAWDRN
GQDLTAHFTTSIPLKGNVRNLSVKIRECTGLAFEWWRTVYEKTDLPLVRK
RTISIWGTTLYPQVEDKVEND*
SEQ ID NO.: 28
Deduced amino acid sequence of domain 4 D385N
W433F (both in bold) with a N-terminal His-tag
(boxed) encoded by pBM90 [symbol]
GDLLLDHSGAYVAQ
YYITWDELSYNHQGKEVLTPKAWDRNGQDLTAHFTTSIPLKGNVRNLSVK
IRECTGLAFEWWRTVYEKTDLPLVRKRTISIWGTTLYPQVEDKVEND*
SEQ ID NO.: 29
Deduced amino acid sequence of domain 4 D385N
W433F (both in bold) encoded by pBM91
MGDLLLDHSGAYVAQYYITWDELSYNHQKGEVLTPKAWDRNGQDLTAHFT
TSIPLKGNVRNLSVKIRECTGLAFEWWRTVYEKTDLPLVRKRTISIWGTT
LYPQVEDKVEND*
SEQ ID NO.: 30
Spn 0001, Forward, NcoI
CATGCCATGGCAAATAAAGCAGTAAATGAC
SEQ ID NO.: 31
Spn0002, Reverse, XhoI
CAGCCGCTCGAGCTAGTCATTTTCTACCTTATCCTC
SEQ ID NO.: 32
Spn0004, Reverse, XhoI
GGCCGCTCGAGCTAGTTTCTGTAAGCTGTAACCTTAGT
SEQ ID NO.: 33
Spn0005, forward, BglII
GGCGGAAGATCTGATGGCAAATAAAGCAGTAAATGAC
SEQ ID NO.: 34
Spn0006, Forward, BglII
GGCGGAAGATCTGGGAGATTTACTGCTGGATCATAG
SEQ ID NO.: 35
Spn0007, Forward, NcoI
CGCATGCCATGGGAGATTTACTGCTGGATCATAG
SEQ ID NO.: 36
Spn0012, non-coding
ACCTTGATGATTATAGGATAATTC
SEQ ID NO.: 37
Spn0013, coding
GAATTATCCTATAATCATCAAGGT
SEQ ID NO.: 38
Spn0015, XhoI
GAGCCGCTCGAGCTATATTTTTTCATACTGCATTCTAGCTGGGACATTAT
TGACCTGACCATAATCTTGATGGTCATTTTCTACCTTATCCTCTACC
SEQ ID NO.: 39
SPN0016, XHOI
GCCGCTCGAGCTACGTTATTTTTTCATACTGCATTCTAGCTGGGACATTA
TTGACCTGACCATAATCTTGATGCCAATAGAGAGTTGTTCCCCAAATAG
SEQ ID NO.40
Spn0048
cgggcttgcctttgaatggtggc
SEQ ID NO.: 41
Spn0049
gccaccattcaaaggcaagcccg

What is claimed is:

1. A recombinant DNA molecule encoding a polypeptide consisting essentially of one domain of pneumolysin joined to a PLY4 antigen.

2. The recombinant DNA molecule of claim 1 wherein the domain of pneumolysin is Domain 4.

3. The recombinant DNA molecule of claim 2 wherein Domain 4 has the sequence shown in SEQ ID NO.:X.

4. The recombinant DNA molecule of claim 2 wherein Domain 4 comprises at least one mutation. selected from the group consisting of His367Arg, Asp385Asn, Cys428Gly, and Trp433Phe.

5. The recombinant DNA molecule of claim 2 wherein Domain 4 comprises the mutation Asp385Ans.

6. The recombinant DNA molecule of any one of claims 1-5 wherein the codon sequence differs from that of pneumolysin shown in SEQ ID NO.: 1 due to the degeneracy of the genetic code.

7. The recombinant DNA molecule of any one of claims 1-6 wherein at least one amino acid residue of the polypeptide is conservatively substituted by a different amino acid residue

8. The recombinant DNA molecule of any one of claims 1-7 formulated as a pharmaceutically acceptable composition.

9. A polypeptide encoded by a DNA molecule of any one of claims 1-8.

10. The polypeptide of claim 9 formulated as a pharmaceutically acceptable composition.

11. A recombinant DNA molecule encoding a polypeptide consisting essentially of Domain 4 of pneumolysin. formulated as a pharmaceutically acceptable composition.

12. The recombinant DNA molecule of claim 11 wherein Domain 4 comprises a mutation selected from the group consisting of His367Arg, Asp385Asn, Cys428Gly, and Trp433Phe.

13. The recombinant DNA molecule of claim 11 wherein Domain 4 comprises the mutation Asp385Asn.

14. A recombinant DNA molecule of any one of claims 1-8 or 11-13 formulated as a pharmaceutically acceptable solution and comprising an adjuvant.

15. A polypeptide of claim 9 or 10 further comprising an adjuvant.

16. A method of generating immunity against S. pneumoniae comprising administering a recombinant DNA molecule of any one of claims 1-8 or 11-13 to a host.

17. A method of generating immunity against S. pneumoniae comprising administering a polypeptide of claim 9 or 10 to a host.

18. A method of claim 16 or 17 wherein administration is via an intravenous, intradermal, subcutaneous, oral, or mucosal route.


SEQUENCE LISTING (41 sequences)

SEQ ID NO: 1 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 2 - protein, 507 aa, Artificial
  1-36 His tag; 37-507 Streptococcus pneumoniae
MHHHHHHSSGLVPRGSGMKETAAAKFERQHMDSPDLMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 3 - protein, 147 aa, Artificial
  1-36 His tag; 37-147 Streptococcus pneumoniae
MHHHHHHSSGLVPRGSGMKETAAAKFERQHMDSPDLGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 4 - protein, 112 aa, Streptococcus pneumoniae
MGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 5 - protein, 396 aa, Artificial
  1-36 His tag; 37-396 Streptococcus pneumoniae
MHHHHHHSSGLVPRGSGMKETAAAKFERQHMDSPDLMANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRN

SEQ ID NO: 6 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 7 - protein, 131 aa, Artificial
  Streptococcus pneumoniae
MGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDHQDYGQVNNVPARMQYEKI

SEQ ID NO: 8 - protein, 166 aa, Artificial
  1-36 His tag; 37-166 Streptococcus pneumoniae
MHHHHHHSSGLVPRGSGMKETAAAKFERQHMDSPDLGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVENDHQDYGQVNNVPARMQYEKI

SEQ ID NO: 9 - protein, 123 aa, Artificial
  Streptococcus pneumoniae
MGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYWHQDYGQVNNVPARMQYEKIT

SEQ ID NO: 10 - protein, 158 aa, Artificial
  1-36 His tag; 37-158 Streptococcus pneumoniae
MHHHHHHSSGLVPRGSGMKETAAAKFERQHMDSPDLGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYWHQDYGQVNNVPARMQYEKIT

SEQ ID NO: 11 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYLHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 12 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYEHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 13 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 14 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLALEWWRTVYEKTDLPLVRKRTISIWGTTLYPQVEDKVEND

SEQ ID NO: 15 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 16 - protein, 471 aa, Artificial
  Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYNHQGKEVLTPKAWDRNGQDLTAHFTTSIPLKGNVRNLSVKIRECTGLAFERWRTVYEKTDLPLVRKRTISIWGTTLYPQVEDKVEND

SEQ ID NO: 17 - protein, 131 aa, Artificial; identical to SEQ ID NO: 7
  Streptococcus pneumoniae

SEQ ID NO: 18 - protein, 132 aa, Artificial
  Streptococcus pneumoniae
GDLLLDHSGAYVAQYYITWDELSYEHQGKEVLTPKAWDRNGQDLTAHFTTSIPLKGNVRNLSVKIRECTGLAWEWWRTVYEKTDLPLVRKRTISIWGTTLYPQVEDKVENDWHQDYGQVNNVPARMQYEKIT

SEQ ID NO: 19 - protein, 132 aa, Streptococcus pneumoniae
GDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVENDWHQDYGQVNNVPARMQYEKIT

SEQ ID NO: 20 - protein, 132 aa, Artificial
  Streptococcus pneumoniae
GDLLLDHSGAYVAQYYITWDELSYNHQGKEVLTPKAWDRNGQDLTAHFTTSIPLKGNVRNLSVKIRECTGLALEWWRTVYEKTDLPLVRKRTISIWGTTLYPQVEDKVENDWHQDYGQVNNVPARMQYEKIT

SEQ ID NO: 21 - protein, 132 aa, Artificial
  Streptococcus pneumoniae
GDLLLDHSGAYVAQYYITWDELSYNHQGKEVLTPKAWDRNGQDLTAHFTTSIPLKGNVRNLSVKIRECTGLAFEWWRTVYEKTDLPLVRKRTISIWGTTLYPQVEDKVENDWHQDYGQVNNVPARMQYEKIT

SEQ ID NO: 22 - protein, 131 aa, Artificial
  Streptococcus pneumoniae
GDLLLDHSGAYVAQYYITWDELSYNHQGKEVLTPKAWDRNGQDLTAHFTTSIPLKGNVRNLSVKIRECTGLAFERWRTVYEKTDLPLVRKRTISIWGTTLYPQVEDKVENDWHQDYGQVNNVPARMQYEKI

SEQ ID NO: 23 - protein, 147 aa, Artificial
  1-36 His tag; 37-147 Streptococcus pneumoniae
MHHHHHHSSGLVPRGSGMKETAAAKFERQHMDSPDLGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 24 - protein, 112 aa, Artificial
  Streptococcus pneumoniae
MGDLLLDHSGAYVAQYYITWDELSYNHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 25 - protein, 112 aa, Artificial; identical to SEQ ID NO: 24
  Streptococcus pneumoniae

SEQ ID NO: 26 - protein, 471 aa, Artificial; identical to SEQ ID NO: 13
  Streptococcus pneumoniae

SEQ ID NO: 27 - protein, 471 aa, Artificial; identical to SEQ ID NO: 15
  Streptococcus pneumoniae

SEQ ID NO: 28 - protein, 147 aa, Artificial
  1-36 His tag; 37-147 Streptococcus pneumoniae
MHHHHHHSSGLVPRGSGMKETAAAKFERQHMDSPDLGDLLLDHSGAYVAQYYITWDELSYNHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 29 - protein, 112 aa, Artificial
  Streptococcus pneumoniae
MGDLLLDHSGAYVAQYYITWDELSYNHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 30 - DNA, 30 bp, Artificial
  Streptococcus pneumoniae
catgccatggcaaataaagcagtaaatgac

SEQ ID NO: 31 - DNA, 36 bp, Artificial
  Streptococcus pneumoniae
cagccgctcgagctagtcattttctaccttatcctc

SEQ ID NO: 32 - DNA, 38 bp, Artificial
  Streptococcus pneumoniae
ggccgctcgagctagtttctgtaagctgtaaccttagt

SEQ ID NO: 33 - DNA, 37 bp, Artificial
  Streptococcus pneumoniae
ggcggaagatctgatggcaaataaagcagtaaatgac

SEQ ID NO: 34 - DNA, 36 bp, Artificial
  Streptococcus pneumoniae
ggcggaagatctgggagatttactgctggatcatag

SEQ ID NO: 35 - DNA, 34 bp, Artificial
  Streptococcus pneumoniae
cgcatgccatgggagatttactgctggatcatag

SEQ ID NO: 36 - DNA, 24 bp, Artificial
  Streptococcus pneumoniae
accttgatgattataggataattc

SEQ ID NO: 37 - DNA, 24 bp, Artificial
  Streptococcus pneumoniae
gaattatcctataatcatcaaggt

SEQ ID NO: 38 - DNA, 97 bp, Artificial
  Streptococcus pneumoniae
gagccgctcgagctatattttttcatactgcattctagctgggacattattgacctgaccataatcttgatggtcattttctaccttatcctctacc

SEQ ID NO: 39 - DNA, 99 bp, Artificial
  Streptococcus pneumoniae
gccgctcgagctacgttattttttcatactgcattctagctgggacattattgacctgaccataatcttgatgccaatagagagttgttccccaaatag

SEQ ID NO: 40 - DNA, 23 bp, Artificial
  Streptococcus pneumoniae
cgggcttgcctttgaatggtggc

SEQ ID NO: 41 - DNA, 23 bp, Artificial
  Streptococcus pneumoniae
gccaccattcaaaggcaagcccg